(19) United States
(12) Patent Application Publication
(10) Pub. No.: US 2018/0161568 A1
(43) Pub. Date: Jun. 14, 2018

(54) STERILE FLUID CONNECTION

(71) Applicant: Parker-Hannifin Corporation, Cleveland, OH (US)

(72) Inventors: Gino G. Banco, Lyndhurst, OH (US); Andrew D. Sylvester, Richmond, VA (US)

(21) Appl. No.: 15/738,608

(22) PCT Filed: Jul. 15, 2016
(86) PCT No.: PCT/US2016/042562

§ 371 Date: Dec. 21, 2017

Related U.S. Application Data
(60) Provisional application No. 62/193,654, filed on Jul. 17, 2015.

(51) Int. Cl. A61M 39/18; A61M 39/26

(52) U.S. Cl. CPC A61M 39/18; A61M 2039/1094; A61M 2039/265; A61M 39/26

(57) ABSTRACT

A fluid connection device includes a first connector having at least one fluid passage and a second connector having at least one fluid passage. The second connector includes a sealing door that may be opened and closed for sealing the fluid passage when the first connector and the second connector are disconnected. The first connector includes a mechanical or electronic key for actuating the second connector, such that the sealing door opens in response to the key. When the sealing door is opened, the first connector may be joined with the second connector. When the first connector is fully joined with the second connector, the fluid passages may be adjacently aligned and in fluid communication for fluid flow therebetween.

### FIELD OF INVENTION

The present invention relates generally to fluid connection devices, and more particularly to fluid connection devices that may be connected and disconnected.

### BACKGROUND OF THE INVENTION

Fluid connection devices may be implemented in a variety of applications where fluid flows from one location to another. Examples of suitable applications for a fluid connection device may be found in the medical field. Generally, fluid lines are used to deliver various fluids between two entities. For example, fluid lines may be used to deliver medicine to a patient, to draw blood from the patient, or to connect a fluid dispensing device to a tube set for delivery.

Conventional fluid connection devices generally include a male connection end that is insertable into a complementary female connection end. However, conventional male and female connectors may be deficient in that the connectors may not be configured to provide a sanitary or aseptic connection, allowing the fluid to become contaminated from bacteria, viruses, or other microorganisms. A particular issue with contamination in medical connection devices is associated with when the two connection ends are disconnected. Biological material can leak during disconnection which can present a serious biohazard.

### SUMMARY OF THE INVENTION

The present application is directed towards a fluid connection device that includes a first connector having a key portion that is engageable with an actuatable portion of a second connector. The fluid connection device maintains a sanitary, tamper-resistant connection between the first and second connector, and the second connector may reseal itself when the first connector is disconnected from the second connector.

A fluid connection device may include a first connector having at least one fluid passage, a second connector having at least one fluid passage, the second connector initially being in a closed position, and a key associated with the first connector for actuating the second connector. The second connector may be moveable to an open position when actuated by the key, and the first connector may be insertable into or otherwise joined with the second connector when the second connector is in the open position. The at least one fluid passage of the second connector and the at least one fluid passage of the first connector may be adjacently aligned and in fluid communication when the first connector is fully inserted into or joined with the second connector.

A fluid connection device may include a first connector having at least one fluid passage, a second connector having a housing, at least one fluid passage contained within the housing, and a receiving chamber contained within the housing for receiving the first connector, the receiving chamber being in fluid communication with the at least one fluid passage and having at least one sealing door that may be opened and closed, and a key associated with the first connector for opening the sealing door of the receiving chamber. The sealing door of the receiving chamber may be opened when actuated by the key and the first connector may be inserted in the receiving chamber when the sealing door is opened. The at least one fluid passage of the second connector and the at least one fluid passage of the first connector may be in fluid communication when the sealing door is opened and the first connector is fully inserted in the receiving chamber.

A method of providing fluid communication between fluid passages in a first connector and a second connector may include arranging the second connector in an initially closed position, the second connector having a sealing door. The method may further include actuating the second connector using a key associated with the first connector, wherein the sealing door of the second connector opens in response to recognition of the key. The method may still further include inserting the first connector in the second connector, or otherwise joining the first connector with the second connector, when the sealing door is opened, wherein the fluid passages are in fluid communication when the first connector is fully inserted in or joined with the second connector, when the second connector is in an open position.

### BRIEF DESCRIPTION OF THE DRAWINGS

FIG. 1A is a perspective view of a fluid connection device showing a male connector connected to a female connector in accordance with an exemplary embodiment.

FIG. 1B is a side sectional view of the male connector of FIG. 1A showing the internal features of the male connector.

FIG. 1C is a side sectional view of the male connector of FIG. 1A.

FIG. 1D is a side sectional view of the female connector of FIG. 1A showing the internal features of the female connector.

FIG. 1E is a side sectional view of the female connector of FIG. 1B.

FIG. 2A is a side perspective view of the fluid connection device of FIG. 1A, with the connectors disconnected.

FIG. 2B is a detailed drawing of the female connector of FIG. 2A.

FIG. 2C is a side perspective view of the fluid connection device of FIG. 2A showing the male connector engaging the female connector.

FIG. 3A is a rear perspective view of the fluid connection device of FIG. 1A showing the female connector in an initial position.

FIG. 3B is a rear perspective view of the fluid connection device of FIG. 1A showing the female connector in an open position.

FIG. 3C is a rear perspective view of the fluid connection device of FIG. 1A showing the female connector in a position for receiving the male connector.

FIG. 3D is a rear perspective view of the fluid connection device of FIG. 1A showing the male connector engaging the female connector.

FIG. 3E is a rear perspective view of the fluid connection device of FIG. 1 A showing the male connector connected to the female connector.

FIG. 4 is a side perspective view of a fluid connection device in accordance with a second exemplary embodiment.

FIG. 5A is a schematic drawing of a fluid connection device having a male connector and a female connector in accordance with a third exemplary embodiment.

FIG. 5B is a schematic drawing of the fluid connection device of FIG. 5A showing the male connector connected to the female connector.

### DETAILED DESCRIPTION

The principles of the present application have particular application to connecting fluid lines. More particularly, the present application is directed towards connecting fluid lines used in medical applications. Other suitable applications may include any application that uses hydraulic lines that are to be connected for delivering fluid between separate entities, such as fluid sources. The fluid connection device may include a first connector having at least one fluid passage and a second connector having at least one fluid passage that is initially in a closed position. The first connector includes a key portion that may be inserted in the second connector to actuate the second connector and move the second connector to an open position, such that the first connector may be joined with the second connector, and the fluid passages may be in fluid communication with one another. In the exemplary embodiments described herein, the first connector may be a male connector and the second connector may be a female connector and the male connector may be inserted into the female connector, or the fluid connection device may be configured such that the first connector may be a female connector and the second connector may be a male connector, or the connectors otherwise may be joined together with the key portion being located on one of the connectors and being configured to actuate the other of the connectors when the connectors are joined together.

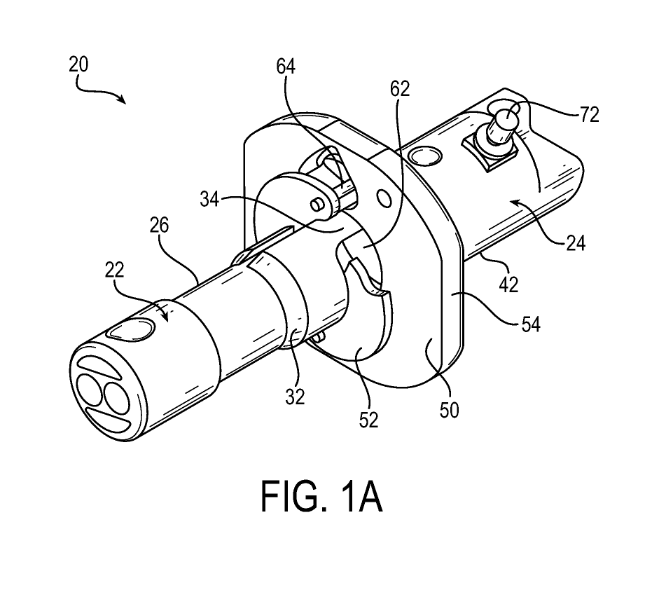

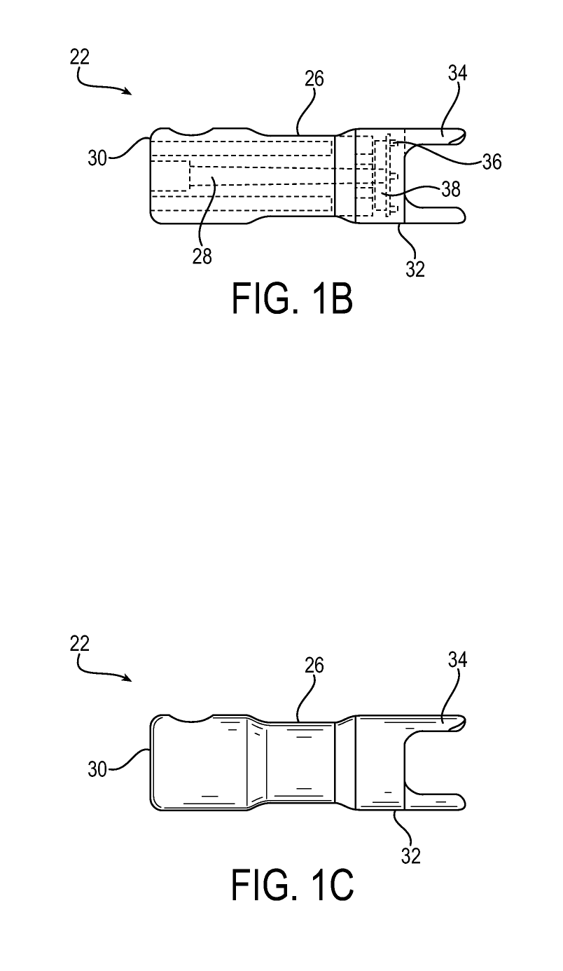

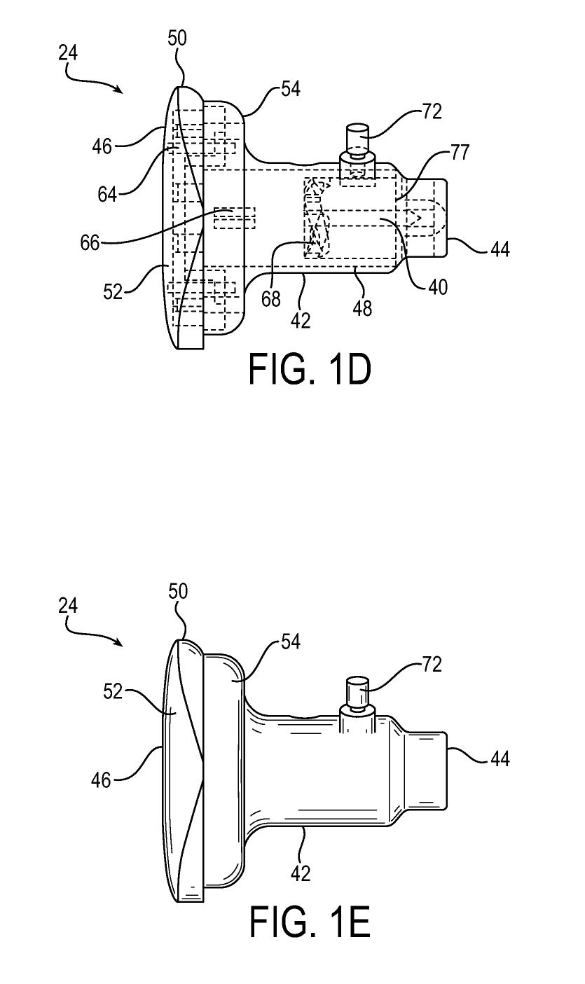

Referring now to FIGS. 1A-3E, a fluid connection device 20 may generally include two separate bodies through which a fluid may flow. The fluid connection device 20 may include a first or male connector 22 and a second or female connector 24 that each have at least one fluid passage. The first and second connectors may be joined together to form a single fluid passage. For example as shown in FIG. 1A, when the second connector is a female connector and the first connector is a male connector, the female connector 24 may be configured to receive the male connector 22 and fluid may flow between the connectors 22, 24 when the connectors 22, 24 are connected.

As shown in FIG. 1B, the male connector 22 may include a body 26 that surrounds at least one fluid passage 28. The male connector 22 may include a plurality of fluid passages. The body 26 of the male connector 22 may have any suitable shape. For example, the body 26 may be substantially cylindrical such that the body 26 circumscribes the fluid passage 28. The fluid passage 28 may extend along a longitudinal axis of the body 26 and the fluid passage 28 may extend through the entire length of the body 26. In an exemplary embodiment where the male connector 22 contains a plurality of fluid passages, the fluid passages may extend parallel to one another throughout the entire length of the body 26. The fluid passage 28 may extend between a first end 30 of the body 26 and a second end portion 32 located distally opposite the first end 30. The first end 30 may be in fluid communication with a fluid source or any suitable entity that may receive fluid or supply fluid. The second end portion 32 of the body 26 may be insertable into the female connector 24. The second end portion 32 may have a different diameter than the diameter of the main body portion of the body 26.

Referring in addition to FIG. 1C, the male connector 22 may include at least one key portion or key 34 that extends from the body 26 of the male connector 22. The male connector 22 may include a plurality of key portions. The plurality of key portions may be spaced apart and the key portions may be circumferentially spaced around the body 26. The at least one key 34 may extend axially outwardly from the second end portion 32 of the body 26, such that the key 34 extends towards the female connector 24. The key 34 may be in the form of a flange or axially extending arm of the second end portion 32. The body 26, the second end portion 32, and the at least one key 34 may be formed as a continuous part such that the male connector 22 is formed as a unitary body.

The male connector 22 may further include at least one locking mechanism 36 (FIG. 1B) that is engageable with a corresponding locking mechanism of the female connector 24 for preventing rotational and axial movement of the male connector 22 relative to the female connector 24. The locking mechanism 36 may be in the form of a lock tab 36 or a plurality of lock tabs. The locking mechanism 36 may be positioned within the body 26 of the male connector 22 or within the second end portion 32 of the body 26, as shown in FIG. 1B. The locking mechanism 36 may be defined on an interior surface of the second end portion 32. The locking mechanism may include any suitable mechanism that is engageable with a corresponding mechanism of the female connector 24, such as protrusions, prongs, pins, posts, notches, catches, and flexible tabs. In an exemplary embodiment, the locking mechanism may be disposed on an exterior portion of the body 26 or the second end portion 32 of the body 26. Many other locking mechanisms may be suitable for locking the male connector 22 and the female connector 24. The plurality of lock tabs 36 may be circumferentially spaced about the longitudinal axis of the male connector 22.

The male connector 22 may further include a sealing member 38 as shown in FIG. 1B. The sealing member 38 may be disposed within the second end portion 32 and adjacent the at least one lock tab 36. The sealing member 38 may be formed of any suitable material and may extend radially from the longitudinal axis of the fluid passage 28. The sealing member 38 may be in the form of a gasket or any other suitable seal for sealing fluid communication between adjacent fluid passages.

Referring now to FIG. 1D, the female connector 24 may include at least one fluid passage 40 contained within a housing 42 of the female connector 24. The female connector 24 may include a plurality of fluid passages. The fluid passage 40 may be fluidly connectable with the fluid passage 28 of the male connector 22 when the housing 42 receives the male connector 22. The male connector 22 and the female connector 24 may include a same or different number of fluid passages having various dimensions. The fluid connection device 20 may be configured to seal fluid communication between a plurality of fluid passages in one of the connectors with a single fluid passage in the other of the connectors. The connectors may be multi-lumen, such that they may have multiple fluid paths arranged in parallel or in any suitable pattern. In an exemplary embodiment, a gasket may be provided for aligning the fluid pathways, such that the paths are sealed. The gasket may be formed directly on the male connector or fixed to the male connector. Each fluid path on the gasket may have a raised feature, such as an o-ring face seal, for creating a seal around the path.

The housing 42 of the female connector 24 may have any suitable shape. For example, the housing 42 may be substantially cylindrical such that the housing 42 circumscribes the fluid passage 40 and the fluid passage 40 extends along a longitudinal axis of the female connector 24. The fluid passage 40 may extend from a first end 44 of the housing 42 towards a second end 46 located distally opposite the first end 44. The fluid passage 40 may be in fluid communication through the first end 44 with a fluid source or any suitable entity that may receive fluid or supply fluid. The fluid passage 40 may extend from the first end 44 to a location midway through the housing 42, such that the fluid passage 40 does not extend through the entire length of the housing 42. The housing 42 may include a receiving chamber 48 for receiving the second end portion 32 of the male connector 22. The receiving chamber 48 may be defined by an interior wall within the housing 42 of the female connector 24 and the receiving chamber 48 may have a shape that is complementary to the shape of the male connector 22.

Referring in addition to FIGS. 1A and 1E, the female connector 24 may further include a flap body portion 50 located at the second end 46 of the housing 42. The flap body portion 50 may have a different diameter than the diameter of the housing 42. In an exemplary embodiment, the diameter of the flap body portion 50 may be greater than the diameter of the housing 42. The flap body portion 50 may support at least one flap door 52 or a plurality of flap doors that are mounted to the flap body portion 50. As shown, the female connector 24 may include at least two flap doors 52. The flap doors 52 may be rotatable about the longitudinal axis of the fluid passages and axially moveable along the longitudinal axis to open the female connector 24. The flap doors 52 may move in unison, such that the flap doors 52 both move towards or away from the female connector 24 or towards and away from the longitudinal axis.

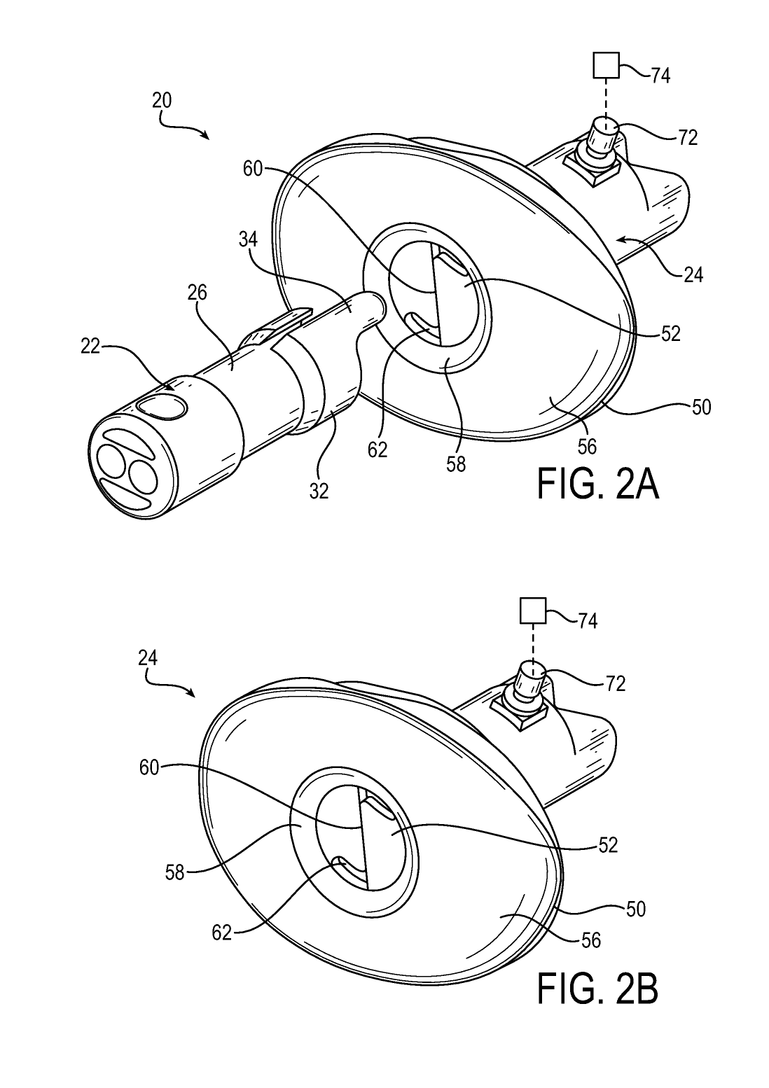

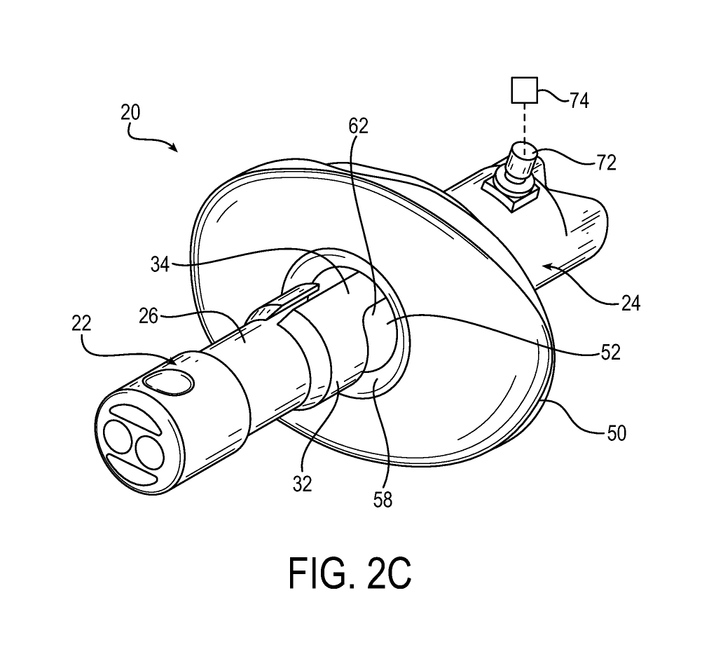

Referring in addition to FIGS. 2A-2C, the female connector 24 may initially be in a closed position as shown in FIGS. 2A and 2B, where the flap doors 52 are engaged with one another to close the female connector 24. When the female connector 24 is actuated by the key 34 of the male connector 22, the flap doors 52 may be rotated and/or axially moved to separate from one another and move the female connector 24 towards an open position for receiving the male connector 22. The opening of the flap doors in shown in a progression from FIG. 2C in which the two connectors come in contact, to the file joined position of the connection device as shown in FIG. 1A. The flap doors 52 may rest against the flap body portion 50 when the female connector 24 is in the closed position. The flap doors 52 ensure that the female connector 24 is self-sealing or remains sealed when the male connector 22 and the female connector 24 are disconnected, such that contaminants are prevented from entering the fluid passage 40 of the female connector 24.

The flap body portion 50 may have any suitable configuration for supporting the flap doors 52, and the configuration of the flap body portion 50 may be dependent on the configuration of the flap doors 52. As shown in FIG. 1A, the flap body portion 50 may include a mounting wall 54 that supports the flap doors 52. The mounting wall 54 may be contained within an exterior body 56, as shown in FIGS. 2A-2C. The flap doors 52 may be complementary in shape and may be positioned adjacently to one another when the female connector 24 is in the closed position. The flap doors 52 may be received within a circular recess 58 defined by the exterior body 56. As shown in FIGS. 2A and 2B, the flap doors 52 may each have an engaging surface 60 such that the flap doors 52 are engageable along the respective engaging surface 60. The flap doors 52 may be axially offset from one another along the engaging surface 60 such that at least one key-receiving aperture 62 is formed between the flap doors 52 and the circular recess 58. As shown in FIGS. 2A-2C, at least two key-receiving apertures 62 may be formed. Due to a smaller diameter of the fluid passage 40, the fluid passage 40 may remain sealed via the engaging surfaces 60 preventing contaminants from entering any opening of the fluid passage 40 located behind the engaging surfaces 60.

The female connector 24 may be actuated by the key 34 of the male connector 22 being inserted in the key-receiving aperture 62. As best shown in FIG. 2C, the key-receiving aperture 62 may have a shape that is complementary to the shape of the key 34. After the key 34 has been inserted into the key-receiving aperture 62, the flap doors 52 may be moved away from another by either the key 34 being rotated or by the key 34 being inserted further into the key-receiving aperture 62. As shown in FIGS. 1A and 1D, the flap doors 52 may be fixed to support posts 64 that enable the flap doors 52 to move apart from one another as the key 34 is inserted further into the female connector 24. The support posts 64 may be supported by a feature within the mounting wall 54 or the flap body portion 50 within the female connector 24. The mounting wall 54 may include an internal guiding mechanism along which the support post 64 may move to move the flap door 52. The female connector 24 is actuated by the key 34 such that the female connector 24 moves from the closed position shown in FIGS. 2A and 2B to the open position shown in FIG. 2C, allowing the body of the male connector 22 to be fully inserted within the female connector 24 as shown in the final joined position of FIG. 1A. The flap doors 52 may be normally closed to reduce tampering. The flap doors 52 may be spring loaded with torsion springs, such that the flap doors 52 will return to the closed position when the key 34 of the male connector 22 is removed from the female connector 24.

When the female connector 24 has been actuated into the open position, the second end portion 32 of the male connector 22 may be fully inserted within the receiving chamber 48 of the female connector 24. The male connector 22 may be rotatable and axially moveable. The male connector 22 may share a longitudinal axis with the receiving chamber 48 such that the male connector 22 rotates about the axis and is axially moveable along the axis. As shown in FIG. 1D, the receiving chamber 48 may further include guide rails 66 defined on walls of the receiving chamber 48 that are engageable with a corresponding feature of the body 26 of the male connector 22. The guide rails 66 are provided for guiding and aligning the fluid passage 28 of the male connector 22 with the fluid passage 40 of the female connector 24 as the male connector 22 moves within the receiving chamber 48. The lock tabs 36 of the male connector 22 may also be engageable with a corresponding locking mechanism 68, shown in FIG. 1D, defined within the receiving chamber 48 of the female connector 24 for preventing further axial movement and rotation of the male connector 22.

The male connector 22 may be fully inserted within the female connector 24 to a predetermined axial position that may be defined by an end wall 67 of the receiving chamber 48, as shown in FIG. 1D. When the male connector 22 has been fully inserted within the female connector 24, the male connector 22 may be further rotated until the lock tabs 36 engage the locking mechanism 68. The locking mechanisms may be engageable via rotation of the male connector 22 in a first direction and disengageable via rotation of the male connector 22 in a second direction opposite the first direction. Rotating the male connector 22 may also actuate the seal member 38 of the male connector 22 and seal the connection between the fluid passages. The seal member 38 may be compressed against a corresponding internal sealing surface 70 of the female connector 24 via rotation of the male connector 22.

The female connector 24 may further include a sensing mechanism 72 for sensing a position of male connector 22. The sensing mechanism 72 may be any suitable position sensor. The sensing mechanism 72 may include a switch and the sensing mechanism 72 may be disposed on an exterior surface of the housing 42 of the female connector 24. As schematically shown in FIGS. 1A and 2A-2C, the sensing mechanism 72 may be in communication with a processor 74 that is configured to receive a signal from the sensing mechanism 72. The sensing mechanism 72 and processor 74 may be configured to detect when the male connector 22 is fully inserted within the female connector 24, or when the male connector 22 has traveled to a predetermined axial position within the female connector 24. The processor 74 may be configured to receive information from the sensing mechanism 72 regarding the position of the male connector 22, and the processor 74 may be configured to determine that the male connector 22 is fully inserted within the female connector 24. The processor 74 may be in communication with a fluid source such that the processor 74 is configured to initiate fluid flow through the fluid passages once the processor 74 has determined that the male connector 22 is fully inserted in the female connector 24. When the fluid passages are connected, fluid may flow in any direction such that fluid may flow from the male connector 22 to the female connector 24 or from the female connector 24 to the male connector 22.

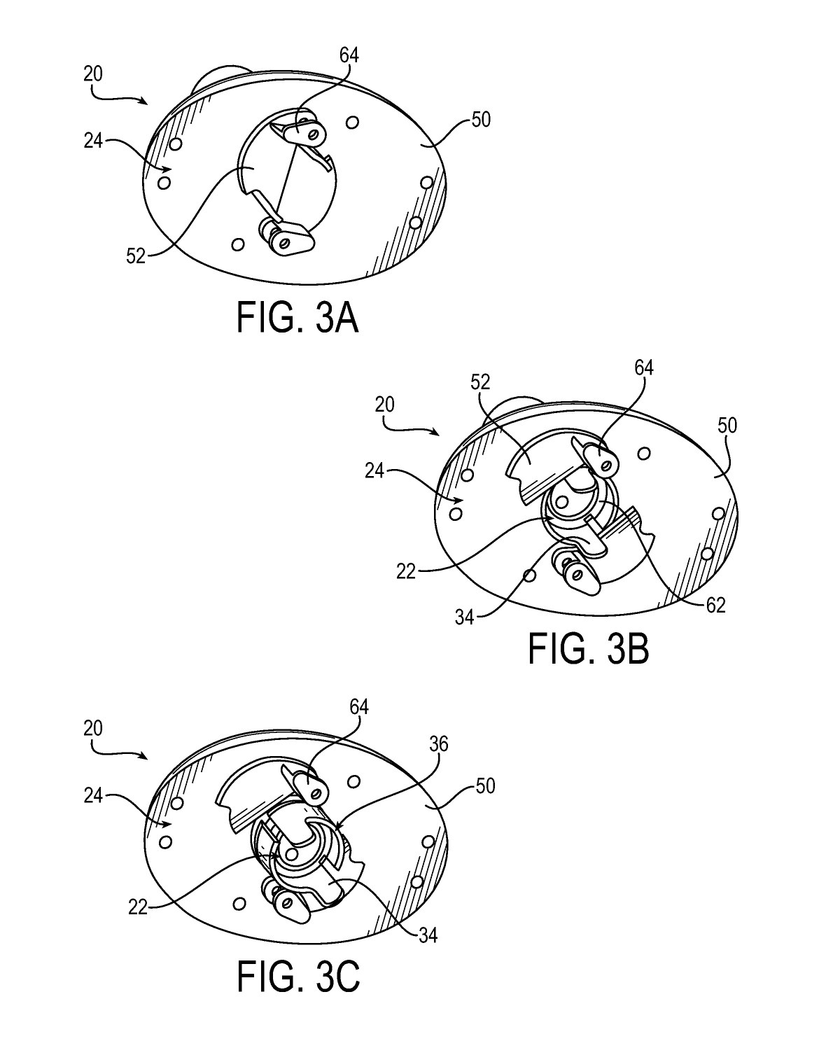

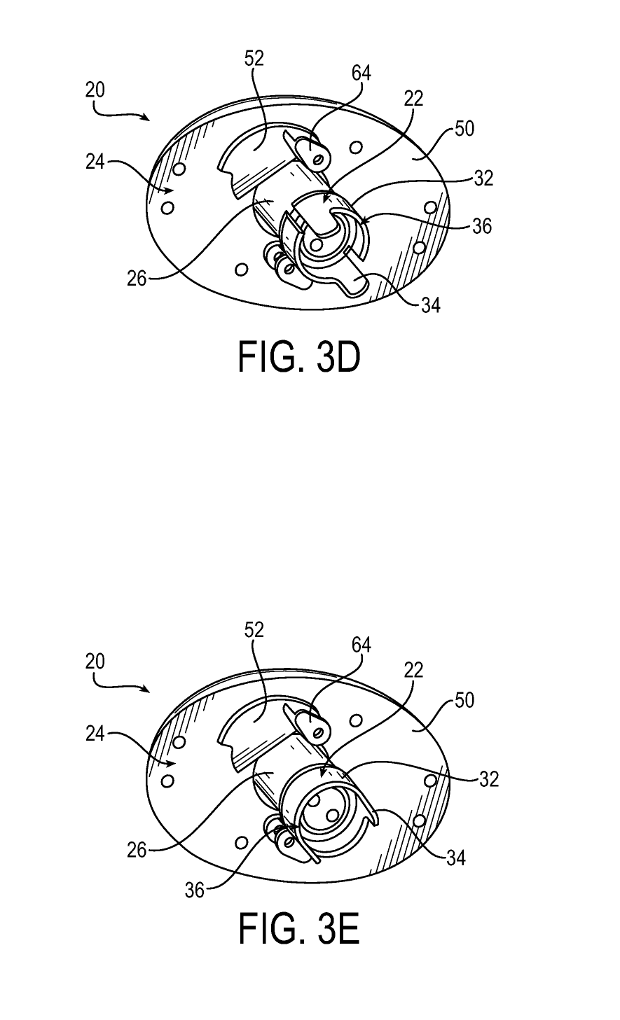

Referring now to FIGS. 3A-3E, a rear perspective view of the fluid connection device 20 (i.e., a view from the female connector side) is shown as the fluid connection device 20 is actuated. FIG. 3A shows the female connector initially in the closed position with the flap doors 52 closed. The flap doors 52 are supported by the flap body portion 50 of the female connector and support posts 64. FIG. 3B shows the flap doors 52 being separated via the insertion of the key 34 of the male connector. The key 34 is inserted through the key-receiving aperture 62 and either rotated or further inserted into the key-receiving aperture 62 to open the flap doors 52. As shown in FIG. 3C, the key 34 may be further rotated such that the lock tabs 36 are rotated until they engage with a corresponding locking mechanism on the female connector 24. As shown in FIGS. 3C-E, the key 34 may be rotated until the second body portion 32 and the body 26 of the male connector are fully received within the female connector. Rotating the key 34 may also enable axial movement of the male connector through the female connector.

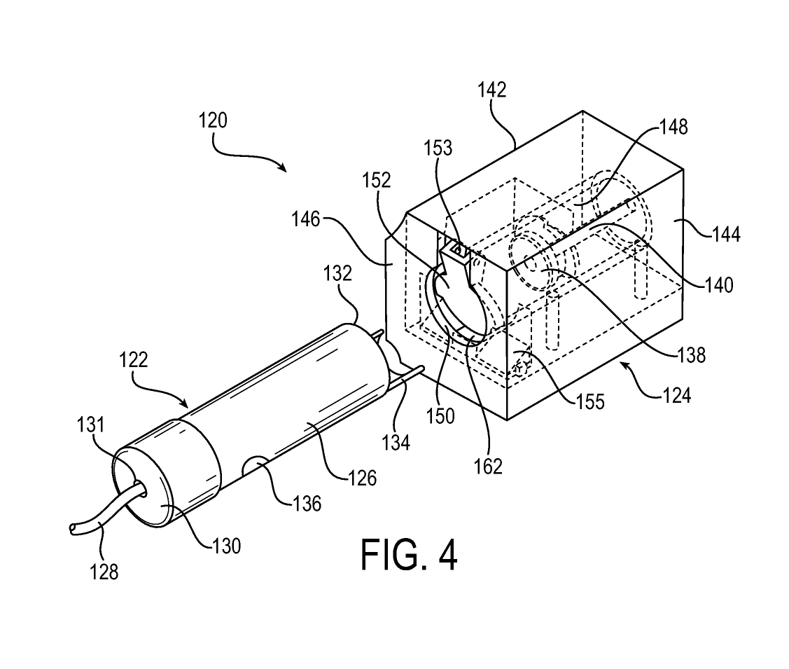

Referring now to FIG. 4, a fluid connection device 120 is shown in accordance with another exemplary embodiment. The fluid connection device 120 includes a male connector 122 and a female connector 124 that each have at least one fluid passage. The male connector 122 may include a body 126 that contains the at least one fluid passage 128. The body 126 may be cylindrical and circumscribe the fluid passage 128 such that the fluid passage 128 extends along a longitudinal axis of the body 126. The body 126 may include a first end 130 and a second end 132 distally opposite the first end 130. The first end 130 may include at least one aperture 131 through which the fluid passage 128 extends such that the fluid passage 128 may be in fluid communication with another fluid receiving or supplying entity. The fluid passage 128 may extend through the entire length of the body 126 between the first end 130 and the second end 132. The body 126 may include at least one key 134 located at the second end 132. The key 134 may extend axially away from the second end 132 and towards the female connector 124. The key 134 may have a hemi-circular shape that has the same diameter as the body 126.

The female connector 124 may include a housing 142 that contains a fluid passage 140. The housing 142 may have any suitable shape, such as a rectangular shape as shown in FIG. 4. The housing 142 may further define a receiving chamber 148 for receiving the male connector 122. The receiving chamber 148 may be cylindrical in shape and have a shape that is complementary to the shape of the body 126 of the male connector 122. The receiving chamber 148 may extend the entire length of the housing 142. The housing 142 may also include a first end 144 and a second end 146 distally opposite the first end 144. The fluid passage 140 may extend through the first end 144 and be in fluid communication with another fluid receiving or supplying entity. The second end 146 may include an opening 150 to the receiving chamber 148 and a flap door 152 that opens and closes the opening 150. The flap door 152 may be hinged to the housing 142 via a fixed hinge 153. The flap door 152 may be pivotable about the hinge 153 to move from a closed position to an open position, enabling the male connector 22 to be inserted into the receiving chamber 148. A key-receiving aperture 162 may be defined between the flap door 152 and the portion of the housing 142 that defines the opening 150. The key-receiving aperture 162 may be complementary in shape to the key 134 of the male connector 122.

The key 134 may be inserted into the key-receiving aperture 162 to open the flap door 152. Inserting the key 134 in the key-receiving aperture 162 may activate a spring-loaded opening mechanism 155. The spring-loaded opening mechanism 155 may be fixedly positioned within the housing 142 and outside the receiving chamber 148. The spring-loaded opening mechanism 155 may be positioned at a location behind the opening 150 and below the receiving chamber 148. The spring-loaded opening mechanism 155 may normally engage the flap door 152 to bias the flap door 152 in the closed position. When the spring-loaded opening mechanism 155 is activated by the key 134, the force against the flap door 152 may overcome the spring force of the spring-loaded opening mechanism 155, allowing the spring-loaded opening mechanism 155 to be moved by the flap door 152 such that the flap door 152 moves to the open position. The spring-loaded opening mechanism 155 may include any suitable type of spring, such as a flat spring or a metal clip. The closed position may be defined by the flap door 152 being perpendicularly arranged relative to the longitudinal axis of the chamber 148. The open position may be defined by the flap door 152 pivoting approximately 90 degrees to a position perpendicular to the closed position and parallel to the longitudinal axis, such that the male connector 122 may be inserted into the receiving chamber 148 through the opening 150.

The male connector 122 may further include a locking mechanism 136, which may be in the form of locking bumps positioned on an exterior surface of the body 126. Bumps are an example and any suitable locking mechanism may be used. When the male connector 122 has been fully inserted within the receiving chamber 148 of the female connector 124, the male connector 122 may be further rotated until the locking bumps 136 engage with a corresponding female locking mechanism 168 to lock the male connector 120 and the female connector 124 together. The female locking mechanism 168 may be in the form of ridges that engage within the locking bumps 136. When the locking features are engaged, further axial movement or rotation of the male connector 122 is prevented relative to the female connector 124. Rotation of the male connector 122 may also compress a sealing member 138 located along the longitudinal axis within the receiving chamber 148 of the female connector 124. A plurality of sealing members may be provided and the sealing member 138 may be in the form of any suitable seal, such as a gasket. Compressing the sealing member 138 enables the fluid passages of the male connector 122 and the female connector 124 to be in sealed fluid communication.

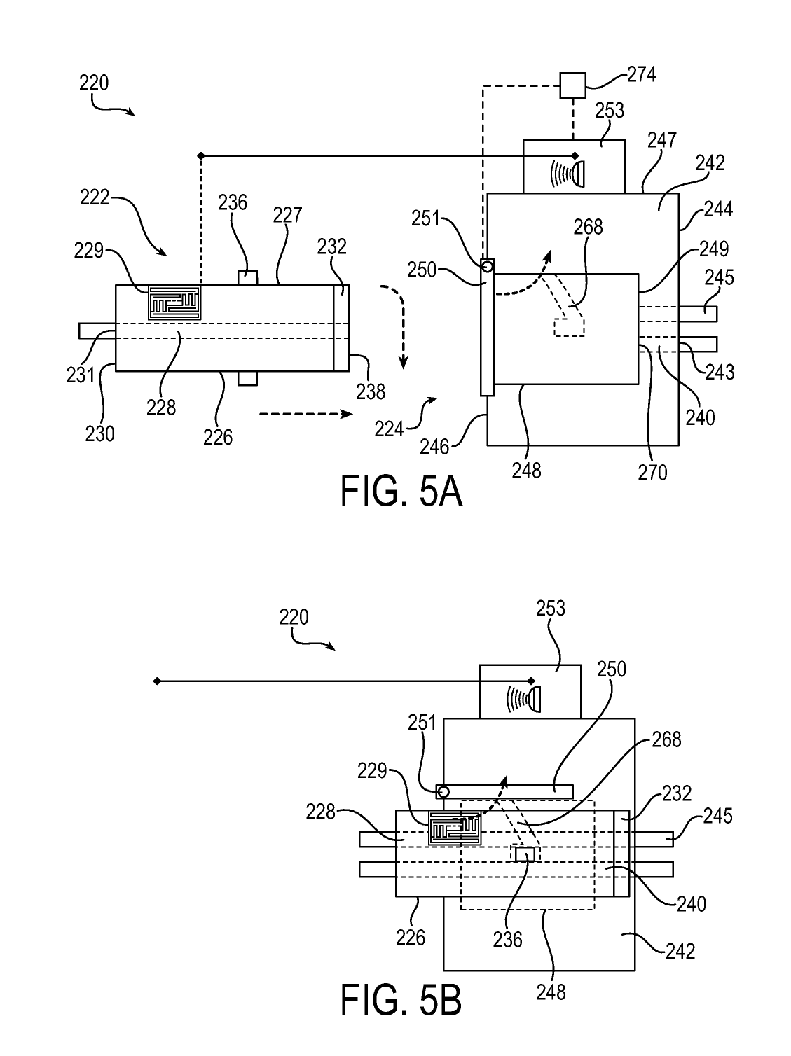

Referring now to FIGS. 5A and 5B, a fluid connection device 220 is shown in accordance with another exemplary embodiment that implements an electronic key for opening the female connector. Using an electronic key may be advantageous in that manufacturing the components may be less complex. Forming the structures of the male connector and the female connector may be less complex due to fewer internal features being used to connect the connectors.

The fluid connection device 220 may include a male connector 222 and a female connector 224 that each include at least one fluid passage or a plurality of fluid passages. The male connector 222 may include a body 226 that defines at least one fluid passage 228. The male connector 222 may include a plurality of fluid passages or channels. The body 226 may have any suitable shape. For example, the body 226 may be rectangular or cylindrical such that the body 226 circumscribes the at least one fluid passage 228. The fluid passage 228 may extend along a longitudinal axis of the body 226 and the fluid passage 228 may be generally centered within the body 226. The body 226 may have a first end 230 and a second end 232 distally opposite the first end 230. The fluid passage 228 may extend through the entire length of the body 226 of the male connector 222 between the first end 230 and the second end 232. The first end 230 may include an aperture 231 through which the fluid passage 228 extends such that the fluid passage 228 may be in fluid communication with a fluid receiving or supplying entity. The body 226 and the fluid passage 228 may have any suitable dimensions and the dimensions may be dependent on the application.

The body 226 may include an electronic identification (ID) chip or tag 229 disposed on an exterior surface thereof. In an exemplary embodiment, the electronic ID tag may be a radio-frequency identification (RFID) tag, but any suitable electronic ID tag may be used. Examples of other suitable ID tags may include Near Field Communication (NFC), Bluetooth, and barcodes or Quick Response (QR) codes. The electronic ID tag 229 may be positioned towards the first end 231 of the body 226 and may be positioned on an upward surface 227 of the body 226. The electronic ID tag 229 may be positioned at any suitable location on or within the body 226. The male connector 222 may further include a seal member 238 disposed at the second end 232 for sealing the fluid passage 228. The second end 232 may be the end of the body 226 that is initially inserted in the female connector 224. The seal member 238 may be annular in shape and may be formed of any suitable material. The seal member 238 may be in the form of any suitable seal with a gasket being just one example.

The female connector 224 may include a housing 242 containing at least one fluid passage. The housing 242 may include a plurality of fluid passages 240. The housing 242 may have any suitable shape, such as a rectangular shape or a cylindrical shape. The housing 242 may include a first end 244 and a second end 246 distally opposite the first end. The first end 244 of the housing 242 may include at least one aperture 243 that is configured to receive a corresponding fluid passage therethrough, such that the fluid passages 240 extend through the housing 242. The fluid passages 240 may be in fluid communication with a fluid supplying or receiving entity. The first end 244 may be engageable with fittings 245 for connecting the fluid passages 240 to the entity. The housing 242 may further include a receiving chamber 248 defined within the housing 242 for receiving the male connector 222 when the body 226 of the male connector 222 is inserted in the female connector 224. The receiving chamber 248 may have a shape that is complementary to the shape of the body 226 of the male connector 222. The fluid passages 240 may be positioned within the housing 242 and outside of the receiving chamber 248. The fluid passages 240 may be adjoined to the receiving chamber 248 at an end wall 249 of the receiving chamber 248. The receiving chamber 248 may further include a sealing surface 270 for sealing the fluid passages 240 to the receiving chamber 248, enabling the fluid passages 228 of the male connector 222 and the fluid passages 240 of the female connector 224 to be sealingly connectable through the end wall 249 of the receiving chamber 248.

The female connector 224 may further include an electronic ID tag reader 253 that is configured to read the electronic ID tag 229 of the male connector 222. The electronic ID tag reader 253 may be in the form of any suitable reader and the form may be dependent on the form of the electronic tag used. In an exemplary embodiment where the electronic ID tag 229 is an RFID tag, the ID electronic tag reader 253 may be an RFID reader. The electronic ID tag reader 253 may be mounted outside of the housing 242. The electronic ID tag reader 253 may be disposed on an upward surface 247 of the housing 242 such that the electronic ID tag reader 253 is positioned to detect a position of the electronic ID tag 229 of the male connector 222 that is disposed on the upward surface 227 of the body 226 of the male connector 222.

The female connector 224 may further include a flap door 250 that is connected to the housing 242 via a hinge 251 that is fixed to the first end 246 of the housing 242. The flap door 250 may be located at the first end 246 of the housing 242 where the male connector 222 is to be inserted into the female connector 224. The fluid connection device 220 may include a controller or processor 274 for controlling movement of the flap door 250. The processor 274 may also be in communication with the electronic ID tag reader 253 for receiving signals from the electronic ID tag reader 253. When the electronic ID tag 229 of the male connector 222 is within a predetermined distance of the electronic ID tag reader 253 of the female connector 224, the electronic ID tag reader 253 may send a signal regarding the position of the male connector 222 to the processor 274. In an exemplary embodiment, the predetermined distance may be between approximately 2.54 centimeters (1 inch) and approximately 91 to 153 centimeters (3 to 5 feet). The processor 274 may be configured to pivot the flap door 250 about the hinge 251 in response to the signal. The flap door 250 may be pivoted from an initially closed position, as shown in FIG. 5A, to an open position for receiving the male connector 222, as shown in FIG. 5B. After the flap door 250 is opened, the male connector 222 may be inserted within the receiving chamber 248 of the female connector 224 for connecting the respective fluid passages 228, 240.

The body 226 of the male connector 222 may be rotatable and axially moveable through the receiving chamber 248 of the female connector 224. The receiving chamber 248 may include guide rails or other internal support features for guiding and aligning the fluid passage 228 of the male connector 222 with the fluid passage 240 as the male connector 222 moves axially into the female connector 224. The guide rails may engage the male connector 222 when the male connector 222 is partially inserted within the receiving chamber 248. The male connector 222 may include lock tabs 236 that extend radially outwardly from the body 226. The lock tabs 236 may be engageable with a corresponding locking feature 268 within the female connector 224. The locking feature 268 may be located within the receiving chamber 248. The locking feature 268 may be fixed to the receiving chamber 248 and may be in the form of any suitable locking mechanism that captures the lock tabs 236 of the male connector 224 to prevent further movement of the male connector 224. Examples of a suitable locking feature 268 include a catch, hook, tab, or notch, but many other locking features may be suitable. The body 226 of the male connector 222 may be rotated until the lock tabs 236 engage the locking feature 268, as shown in FIG. 5B. When the lock tabs 236 have engaged the locking feature 268, the male connector 222 may be prevented from further axial movement and rotation. The end wall 249 of the receiving chamber 242 may also prevent further axial movement of the body 226 of the male connector 222.

Referring now to FIG. 5B, when the male connector 222 and the female connector 224 are locked together via the lock tabs 236 and the locking feature 268, the seal member 238 of the male connector 222 may be compressed against the sealing surface 270 of the female connector 224. The body 226 of the male connector 222 may extend through the receiving chamber 248 of the female connector 224. The fluid passages 240 may be sealed to the at least one fluid passage 228 of the male connector 222 when the body 226 is positioned within the receiving chamber 248. When the seal member 238 is compressed against the sealing surface 270, a water-tight seal may be created and the fluid passages of the male connector 222 and the female connector 224 may be aligned for fluid communication. The connection between the fluid passages may be sealed at various locations within the housing 242 of the female connector 224. Fluid flow through the passages may flow from the male connector 222 to the female connector 224 or from the female connector 224 to the male connector 222.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A fluid connection device comprising:
a first connector having at least one fluid passage;
a second connector having at least one fluid passage, the second connector initially being in a closed position; and
a key associated with the first connector for actuating the second connector;
wherein the second connector is moveable to an open position when actuated by the key when the first connector is joined with the second connector, and when the second connector is in the open position the at least one fluid passage of the second connector and the at least one fluid passage of the first connector are adjacently aligned and in fluid communication when the first connector is fully joined with the second connector.

2. The fluid connection device of claim 1, wherein the first connector includes a main body portion that is rotatable about a longitudinal axis, the key extending from the main body portion in a direction parallel to the longitudinal axis, wherein the key is rotatable with the main body portion about the longitudinal axis, the main body portion and the key being axially moveable along the longitudinal axis.

3. (canceled)

4. The fluid connection device of claim 1, wherein the second connector includes an internal spring loaded opening mechanism for biasing the second connector in the closed position, the internal spring loaded opening mechanism being engageable by the key for actuating the second connector and allowing the second connector to move to the open position.

5. The fluid connection device of claim 1, further comprising an RFID reader associated with the second connector, wherein the key is an RFID transmitter that is detectable by the RFID reader within a pre-determined range distance for actuating the second connector.

6. The fluid connection device of any of claim 1, wherein the second connector includes guide rails that are engageable with the first connector for aligning the at least one fluid passage of the first connector with the at least one fluid passage of the second connector as the first connector is inserted into the second connector.

7. The fluid connection device according to claim 1, wherein the second connector includes at least one flap door that is moveable between the closed position and the open position, the flap door being closed to seal the second connector when in the closed position and the flap door being opened to allow the first connector to be fully inserted when in the open position.

8. The fluid connection device according to claim 1, wherein the second connector and the first connector include locking mechanisms that are engageable with one another for locking the connectors when the first connector is fully joined with the second connector, the locking mechanisms preventing rotational and axial movement of the first connector relative to the second connector.

9. The fluid connection device of claim 8, wherein the locking mechanisms are engageable by rotating the first connector within the second connector in a first direction and disengageable by rotating the first connector in a second direction opposite the first direction.

10. The fluid connection device according to claim 1, wherein the second connector and the first connector and include internal seal members, the internal seal member of the first connector being compressible against the internal seal member of the second connector when the first connector is fully joined with the second connector for sealing fluid communication between the at least one fluid passage of the second connector and the at least one fluid passage of the first connector.

11. The fluid connection device according to claim 1, wherein the second connector includes a sensing mechanism for detecting when the first connector is fully joined with the second connector.

12. The fluid connection device according to claim 1, wherein the first connector is a male connector and the second connector is a female connector, the key being associated with the male connector, and the male connector is inserted into the female connector to actuate the female connector via the key, with the connectors in an intermediate position between connection and disconnection.

13. A fluid connection device comprising:
a first connector having at least one fluid passage;
a second connector having a housing, at least one fluid passage contained within the housing, and a receiving chamber contained within the housing for receiving the first connector, the receiving chamber being in fluid communication with the at least one fluid passage and having at least one sealing door that may be opened and closed; and
a key associated with the first connector for opening the sealing door of the receiving chamber;
wherein the sealing door of the receiving chamber is opened when actuated by the key and the first connector is inserted in the receiving chamber when the sealing door is opened, the at least one fluid passage of the second connector and the at least one fluid passage of the first connector being in fluid communication when the sealing door is opened and the first connector is fully inserted in the receiving chamber.

14. The fluid connection device of claim 13 further comprising a locking mechanism for locking the connectors when the first connector is fully inserted in the second connector, the locking mechanism preventing rotational movement of the first connector relative to the second connector, and a sensing mechanism configured to detect when the first connector is fully inserted in the second connector.

15. (canceled)

16. The fluid connection device according to claim 13, wherein the first connector includes a main body and the key is an axially extending flange of the main body, the key being engageable with the second connector for actuating the second connector, wherein the sealing door and the housing define a key-receiving aperture, the key being insertable within the key-receiving aperture to actuate the second connector, and wherein the housing of the second connector includes an internal spring loaded opening mechanism for biasing the sealing door in a closed position, the internal spring loaded opening mechanism being engageable by the key for actuating the second connector and allowing the sealing door to move to an open position.

17-18. (canceled)

19. The fluid connection device of claim 13, further comprising an RFID receiver associated with the second connector, wherein the key is an RFID transmitter detectable by the RFID receiver within a pre-determined range distance for actuating the second connector.

20. (canceled)

21. The fluid connection device of claim 13, wherein the first connector includes an internal seal that is compressible when the first connector is fully inserted in the receiving chamber for sealing fluid communication between the at least one fluid passage of the second connector and the at least one fluid passage of the first connector.

22. (canceled)

23. A method of providing fluid communication between fluid passages in a first connector and a second connector comprising:
arranging the second connector in an initially closed position, the second connector having a sealing door;
actuating the second connector using a key associated with the first connector, wherein the sealing door of the second connector opens in response to recognition of the key; and
joining the first connector with the second connector when the sealing door is opened, wherein the fluid passages are in fluid communication when the first connector is fully joined with the second connector, when the second connector is in an open position.

24. The method according to claim 23 further comprising rotating the key within a portion of the second connector to actuate the second connector, wherein the key is an extended portion of a main body of the first connector.

25. The method according to claim 23 further comprising transmitting an RFID signal associated with the first connector to an RFID reader associated with the second connector, wherein the key is an RFID transmitter detectable by the RFID reader within a pre-determined range distance for actuating the second connector.

26. The method according to claim 23 further comprising actuating a locking mechanism when the first connector is fully joined with the second connector to prevent rotation of the first connector relative to the second connector, and rotating the first connector within the second connector in a first direction to engage the locking mechanism and rotating the first connector in a second direction opposite the first direction to disengage the locking mechanism.

27. (canceled)
